(19) United States
(12) Patent Application Publication
(10) Pub. No.: US 2024/0188820 A1
(43) Pub. Date: Jun. 13, 2024

(54) VIEWING CONE, DEVICE AND METHOD FOR DETECTING THE MOVEMENTS OF THE EYES OF A PATIENT

(71) Applicant: P³LAB, Céroux-Mousty (BE)

(72) Inventors: Pierre Martin Jack Gérard DAYE, Braine-l'alleud (BE); Antoine Hubert Henri Paul Marie POUPPEZ DE KETTENIS DE HOLLAEKEN, Céroux-Mousty (BE)

(21) Appl. No.: 18/285,923

(22) PCT Filed: Mar. 7, 2022
(86) PCT No.: PCT/EP2022/059299

§ 371 Date: Oct. 6, 2023

(30) Foreign Application Priority Data
Apr. 8, 2021 (BE) BE2021/5272

(51) Int. Cl. A61B 3/113; A61B 3/00; A61B 5/00

(52) U.S. Cl. CPC A61B 3/113; A61B 3/005; A61B 3/0091; A61B 5/6803

(57) ABSTRACT

The disclosure relates to a viewing cone for detecting the movement of the eyes of a patient, the viewing cone comprising a chamber having a first end and a second end, a lateral wall bounding the chamber between the first end and the second end, a periphery of an opening intended to be in contact with the forehead of the patient at the first end, and attachment interface for attachment to a processing unit at the second end, the distance between the periphery of the opening and the attachment interface being adjustable to a fixed length by means of one or more spacers that form the lateral wall. The disclosure also relates to a device for detecting the movement of the eyes of a patient, comprising the viewing cone and a method for detecting the movement of the eyes of a patient.

### TECHNICAL FIELD

The present invention relates to a viewing cone, a device for detecting the movements of the eyes of a patient comprising the viewing cone and a method for detecting the movements of the eyes of a patient implementing the device.

### PRIOR ART

The invention relates to the field of identifying eye disorders and/or neurological disorders and to the rehabilitation of the disorders of the vision by analysing the movement of the eyes of a patient.

The document U.S. Pat. No. 8,951,046 describes an arrangement for cognitive testing that eliminates environmental effects. The arrangement comprises a table-top enclosure having a face mask on one wall and housing a computer screen on the opposite wall. The arrangement allows a measurement of the gaze direction made by determining the position of the pupil when a patient presses its face against the mask to eliminate the movement of the head relative to the computer screen.

The document U.S. Pat. No. 9,004,687 describes a neuropsychological testing apparatus involving an eye tracking. The apparatus comprises a helmet adapted to be mounted on the head of a patient, the helmet having a screen positioned at a predetermined distance from the eyes of the patient. The helmet also comprises prisms positioned between the eyes of the patient and the screen so that the prisms direct the light emitted from a point on the screen towards the eyes of the patient.

The disadvantage of these devices is that there is only one possible distance of the face from the computer screen, which does not allow a series of measurements to be taken under different conditions of distance of the face from visual stimuli on the screens.

There is a need for an apparatus that allow to take a series of measurements under different conditions of distance of the face from visual stimuli.

### DESCRIPTION OF THE INVENTION

To this end, the invention proposes a viewing cone for detecting the movement of the eyes of a patient, the viewing cone comprising
A chamber with a first end and a second end,
A lateral wall delimiting the chamber between the first end and the second end,
A periphery of an opening capable of being in contact with the forehead of the patient at the first end,
An attachment interface for attaching a processing unit at the second end,
the distance between the periphery of the opening and the attachment interface can be adjusted to a fixed length.

Preferably, the distance between the periphery of the opening and the attachment interface is adjustable to a fixed length by one or more spacers forming the lateral wall.

In one variant, the viewing cone comprises a single spacer supporting the periphery of the opening at the first end and the attachment interface for attaching the processing unit at the second end.

In one variant, the viewing cone comprises a first spacer supporting the periphery of the opening at the first end and one or more additional spacers joined together, the last spacer supporting the attachment interface for attaching the processing unit at the second end.

In one variant, the spacers form between 5 and 15 cm of lateral wall.

In one embodiment, the viewing cone further comprises one or more through-orifices in the lateral wall, the orifices being adapted to send visual stimuli to different positions in the chamber.

In one variant, the orifices are aligned between the first end and the second end.

In one variant, the viewing cone is worn on the face of the patient.

In one variant, the viewing cone comprises attachment members for attaching the cone to the head of the patient and/or handles.

In one variant, the viewing cone comprises a partition for the vision of each eye and/or a shutter for the vision of one of the eyes.

In one variant, the shutter comprises a removable screen that selectively obturates the vision of one eye or the other.

In one variant, the viewing cone comprises a filter in front of each eye to select a different portion on each eye of the visual stimulus presented by the processing unit.

In one variant, the periphery of the opening can be adapted to the contour of the face of the patient and/or the contour of the eyes and/or the presence of glasses on the face of the patient.

In one embodiment, the viewing cone further comprises a removable cover adapted to obturate the opening, the cover comprising a camera calibration pattern on its side facing the interior of the chamber.

In one variant, the pattern is concave.

In one variant, the viewing cone is a passive assembly.

The invention also relates to a device for detecting the movement of the eyes of a patient comprising
the viewing cone as described above and
a processing unit attached to the attachment interface of the viewing cone, the processing unit comprising cameras for tracking the movement of the eyes and a screen for displaying visual stimuli.

In one variant, the distance between the periphery of the opening and the attachment interface for attaching the processing unit is adjustable to a fixed length by one or more spacers forming the lateral wall.

In one embodiment, the viewing cone comprises a removable cover adapted to obturate the opening, the cover comprising a pattern for calibrating the cameras on its side facing the interior of the chamber.

In one variant, the processing unit comprises the infrared emitters illuminating the chamber.

According to a variant, the device also comprises a support supporting the processing unit, the viewing cone being attached to the processing unit by the attachment interface.

The invention also relates to a method for detecting the movement of the eyes of a patient, comprising the steps of
Supplying the device as described above,
Setting the distance between the periphery of the opening and the attachment interface to a fixed length.

Preferably, during the adjustment step, one or more spacers are used to form the lateral wall.

In one embodiment, the method further comprises a step of calibrating the cameras during which the opening is obturated by a removable cover comprising a camera calibration pattern on its side turned towards the inside of the chamber.

In one embodiment, the calibration pattern is concave.

In one embodiment, the method also comprises a step of stimulating the eyes by sending visual stimuli into the chamber via through orifices in the wall.

The use of the verb “comprise”, its variants and conjugations in this document in no way precludes the presence of elements other than those mentioned. The use in this document of the indefinite article “a”, “an”, or the definite article “the” to introduce an element does not exclude the presence of a plurality of these elements.

The terms “first”, “second”, “third”, etc. are used in this document exclusively to differentiate between different elements, without implying any order between them.

All the preferred embodiments and all the advantages of the viewing cone according to the invention apply mutatis mutandis to the present device and to the method for detecting the movement of the eyes of a patient.

### BRIEF DESCRIPTION OF THE FIGURES

Further characteristics and advantages of the present invention will become apparent from the following detailed description, for the understanding of which reference is made to the appended figures which show:

FIG. 1 shows a schematic view of an example of embodiment of the rear view of the viewing cone;

FIG. 2, a schematic view of an example of embodiment of the viewing cone as seen from the front;

FIG. 3 shows a schematic view of a cover in one example of embodiment;

FIG. 4, a schematic view of an example of embodiment of a rear view of the viewing cone with the cover;

FIG. 5, a schematic view of an example of embodiment of a viewing cone in profile;

FIG. 6 shows a schematic view of an example of embodiment of the processing unit.

The drawings in the figures are not to scale. Similar elements are generally denoted by similar references in the figures. For the purposes of this document, the identical or similar items may bear the same references. Furthermore, the presence of reference numbers or letters in the drawings cannot be regarded as limiting, even when these numbers or letters are indicated in the claims.

### DETAILED DESCRIPTION OF EMBODIMENTS OF THE INVENTION

The invention relates to a viewing cone for detecting the movement of the eyes of a patient. The viewing cone comprises a chamber with a first end and a second end, with a lateral wall delimiting the chamber between the first end and the second end. A periphery of an opening capable of being in contact with the forehead of the patient is at the first end and an attachment interface for attaching a processing unit is at the second end. The distance between the periphery of the opening and the attachment interface can be adjusted to a fixed length. The viewing cone allows a series of measurements to be taken under different conditions of distance from the face in relation to visual stimuli.

FIG. 1 shows a rear view of the viewing cone 10 according to one example of embodiment of the invention. The viewing cone 10 comprises a chamber 12 with a first end 121 and a second end 122. The chamber is delimited by a lateral wall 14 between the first end 121 and the second end 122. At the first end 121, the viewing cone 10 comprises an opening 16 allowing a patient to see inside the chamber 12 and more specifically, as will be described later, to see a processing unit at the second end 122. The opening 16 is circumscribed by a periphery 18 that can be in contact with the forehead of the patient. In other words, the patient presses the top of the periphery 18 of the opening 16 against his forehead so that his gaze enters the chamber 12 through the opening 16. The periphery 18 of the opening 16 can be flat but is preferably concave towards the inside of the chamber 12; this allows the periphery 18 to be well applied to the face of the patient so that the cone 10 blocks the entry of light from the side of the eyes in order to make the inside of the chamber quite dark. The periphery 18 can also comprise a cavity 19 to adapt the periphery 18 to the nose of the patient. At the second end 122 of the chamber, the cone 10 comprises an attachment interface 20 for attaching a processing unit not visible in FIG. 1. So, once the periphery 18 has been applied to the forehead of the patient and surrounds his field of vision, the patient can see through the opening 16 to the processing unit attached to the attachment interface 20.

In order to take series of measurements under different conditions of distance of the face from visual stimuli broadcast by the processing unit, the distance between the periphery 18 of the opening 16 and the attachment interface 20 is adjustable to a fixed length. In other words, the length of the lateral wall 14 delimiting the chamber 12 between the two ends 121, 122 can be adjusted to a desired value and then maintained for a series of measurements. The distance between the face and the processing unit is set beforehand and the viewing cone maintains this distance during a series of measurements. The face is held at a predetermined distance for the measurements. In this way, the depth of the chamber 12 from the first end 121 towards the other end 122 can be adjusted by varying the length of the lateral wall 14; the chosen depth is then retained for the series of measurements. The fact that the length is fixed allows that the calibration and the series of measurements can be carried out in a standardised, predetermined environment with known dimensions. This environment is taken into account in the calibration and testing. The environment is standardised over time and even from one patient to another.

The fixed length of the distance between the periphery and the attachment interface is the length between a point on the periphery and its projection on the attachment interface, in the direction in which the patient is looking. A reference length for the distance between the periphery 18 of the opening 16 and the attachment interface is, for example, that taken from the centre of the periphery 18, in the area of the periphery 18 that applies to the middle of the forehead of the patient, in line with his nose. The fact that the length is fixed allows that a certain fixed, pre-determined distance can be maintained between the periphery and the interface, without it being possible to change it during a test. For example, the cone allows the patient to remain in the same state despite a sudden gesture (which may be the case with a child).

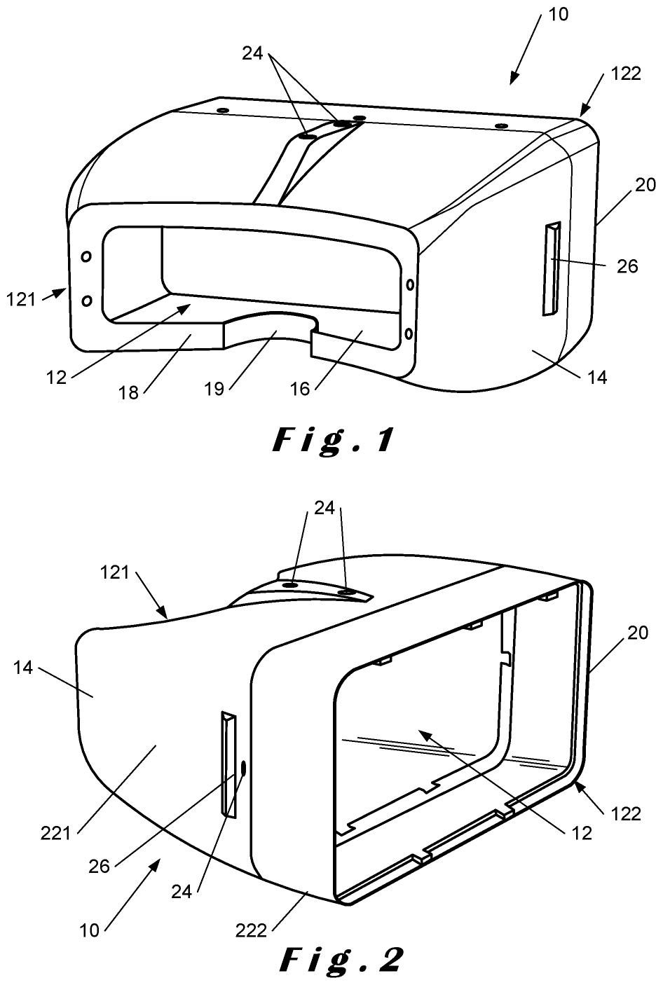

The distance between the periphery 18 of the opening 16 and the attachment interface 20 can be adjusted to a fixed length by one or more spacers forming the lateral wall 14. A spacer is a rigid part that connects two others and keeps them at a fixed distance apart. The choice of the spacer or spacers allows the distance to be set to a fixed length, easily and precisely. The spacers ensure a fixed, constant spacing. The spacers have a fixed length (depending on the direction in which the patient is looking). The spacers have a standardised size (or length, depth), allowing the distance to be adjusted to a fixed length. The spacers have a known, predetermined size (or length, depth), allowing the distance to be set to a fixed length. By assembling one or more spacers, the distance can be adjusted to a fixed length. The distance can be set to a fixed length by adding one or more spacers. By inserting one or more spacers, the distance can be adjusted to a fixed length. The number of spacers required is adjusted to the desired length, so that the cone is not weighed down by unnecessary length-attachment spacers. This allows to make it easy to adjust the depth of the chamber 12 according to the direction in which the patient is looking. In addition, the length of the distance between the periphery 18 of the opening 16 and the attachment interface 20 is repeatable by the spacer or spacers. The use of spacers allows that a certain fixed distance can be maintained between the periphery and the interface, without it being possible to change it during a test. The distance setting cannot be changed during a test. FIG. 2 shows an example of embodiment of how the distance between the periphery 18 of the opening 16 and the attachment interface 20 can be adjusted. Using spacers also allows that you can repeat the same test (or a series of same tests) with the same length (or a series of same lengths) at a later date, because all you have to do is assemble the same spacers. The cone 10 is therefore a set of modules (sub-portions) with known dimensions (standardised modules), which allows it can be adapted to the patients and the tests, and the calibration is also easier.

FIG. 2 shows a front view of the viewing cone 10 of FIG. 1 with the lateral wall 14 delimiting the chamber 12 between the first end 121 and the second end 122. The viewing cone 10 comprises a first spacer 221, forming the base of the viewing cone. The first spacer 221 supports the periphery 18 of the opening 16 at the first end 121 of the viewing cone on one side. In an embodiment in which the viewing cone 10 comprises a single spacer 221, this spacer 221 then also supports the attachment interface 20 for attaching the processing unit at the second end 122. The distance between the periphery 18 of the opening 16 and the attachment interface 20 is set to the minimum fixed length. The depth of the chamber 12 in the direction of the gaze is at a minimum. The same applies to the length of the wall 14 between the first end 121 and the second end 122.

The viewing cone 10 may comprise the first spacer 221 supporting the periphery 18 of the opening 16 at the first end 121 and one or more additional spacers joined together, the last spacer supporting the attachment interface 20 for attaching the processing unit at the second end 122. The second end 122 and the attachment interface 20 are then offset from the first spacer 221 to the last assembled spacer. In this way, the distance between the periphery 18 of the opening 16 and the attachment interface 20 is adjusted to another fixed length depending on the number of additional spacers added to the first spacer 221. FIG. 2 shows an example of the viewing cone 10 in which an additional spacer 222 is assembled to the first spacer 221—the second spacer 222 supporting the attachment interface 20 for attaching the processing unit at the second end 122. The wall 14 is elongated in the direction of the gaze by the length of any additional spacers, a second spacer 222 in FIG. 2 by way of example. The spacers are connected to each other and to the cone by their ends, simply and without bulky attachment means. So, with a set of spacers of different lengths, you can obtain different distances of fixed length.

In this way, the second end 122 and the attachment interface 20 are supported by the last spacer, either the first spacer 221 if there is a single spacer or the N^{th} spacer if N additional spacers are connected to each other and to the first spacer. The distance between the periphery 18 of the opening 16 and the attachment interface 20 is adjustable from a fixed minimum length when a single spacer is used to a greater fixed length when additional spacers are assembled. The depth of the chamber 12 in the direction of the gaze varies according to the number of spacer or spacers used. As the dimensions of these spacers are known, the series of measurements are taken in a standardised, predetermined environment with known dimensions. This environment is taken into account in the calibration and testing.

The additional spacers are attached to each other simply by interlocking and/or clipping. This can be done by the practitioner but also by the patient himself. Each additional spacer attaches to what would have been the attachment interface 20 for attaching the processing unit if the additional spacer had not been used.

The spacers 221, 222, etc. can each form a length of lateral wall 14. The depth of the spacers in the direction of the gaze may vary from one spacer to another or may be similar. In this way, one or more spacers can be used to adjust the distance to a fixed length, by choosing the depth of the spacer or of the spacers. This allows the distance between the periphery 18 of the opening 16 and the attachment interface 20 to be adjusted more finely. For example, the spacers form between 5 and 15 cm of lateral wall 14 in the direction of the gaze. The first spacer 221, forming the base of the viewing cone 10, may form 15 cm of lateral wall 14—this length may correspond to the reference length of the distance between the periphery 18 and the attachment interface 20 taken at the centre of the periphery 18, in the area of the periphery 18 that applies to the middle of the forehead of the patient, in line with his nose. The other spacers can form between 5 cm and 10 cm of lateral wall 14.

The fact that the distance between the periphery 18 of the opening 16 and the attachment interface 20 can be set to a fixed length means that the cone takes up very little space, as it is not necessary to have as many viewing cones as desired distances; the viewing cone 10 is easy to transport and fits in a briefcase. All you need is a set of spacers allowing to adjust the distance.

The viewing cone 10 may further comprise one or more through orifices 24 passing through the lateral wall 14, the orifices 24 being adapted to send visual stimuli to different positions in the chamber 12. The orifices 24 can be seen in FIGS. 1 and 2. This allows to vary the content of the tests and to obtain different measurements in different situations. The number of orifices 24 is not limited. LEDs can be attached to the lateral wall 14 outside the chamber 12 to illuminate the inside of the chamber 12. The light of the LEDs is in visible wavelengths and can be used to test the convergence of the eyes. The orifices 24 can be in multiple positions—for example on at least one of the upper, lower and lateral faces of the lateral wall 14—to create targets in the chamber 12 at different depth planes. The orifices 24 can be aligned between the first end 121 and the second end 122; this allows visual stimuli to be produced at different distances from the face of the patient. According to FIGS. 1 and 2, one or more orifices 24 can be positioned in the lateral wall 14, on the upper or lower side of the lateral wall 14 along a straight line between the area of the periphery 18 which applies to the middle of the forehead of the patient, at right angles to his nose, and the attachment interface 20 in the direction of the gaze. According to FIG. 2, an orifice 24 is located on one of the lateral faces of the lateral wall 14. It is also possible to add other stimulus signals such as sounds.

The viewing cone can be worn on the face of the patient. This means it can be used flexibly to test any patient in any condition. In particular, for patients who are difficult to hold in place during measurements, such as children or babies, the portability of the viewing cone 10 makes it easier to take measurements. The viewing cone is lightweight, making it easy to carry. The viewing cone 10 may comprises attachment members for attaching the cone to the head of the patient and/or handles. The attachment members, not visible in the figures, are for example an adjustable and/or elastic strap extending from the first end 121. The strap passes behind and/or over the head of the patient and is adjusted so that the viewing cone 10 holds its position on his face. Handles allow the patient to hold the viewing cone against their face. Handles 26 on the lateral faces of the lateral wall 14 can be seen in FIGS. 1 and 2.

The viewing cone 10 may comprise a partition for the vision of each eye and/or a shutter for the vision of one of the eyes. This allows various tests to be performed during measurements. To obtain the partition of the vision of each eye, a partition can be placed in the chamber 12 separating the chamber into two fields of vision, one for each eye. For example, a partition can be slid from the end 122 into grooves along the internal surface of the upper and lower faces of the lateral wall 14 in the direction of the gaze, up to the opening 16 against the face of the patient. A shutter can also be placed across the opening 16. The shutter can be slid into a slot in the viewing cone, transverse to the gaze, to lie across the opening 16. The shutter can comprise an obturated area for one eye and an open area for viewing the other eye; the shutter can be changed or interchanged to obturate either eye. The shutter can also comprise a removable screen that selectively obturates the vision of one eye or the other. The screen can be removed or moved across the gaze to obturate the vision in one eye and free up the vision in the other eye.

The viewing cone 10 can also comprise a filter in front of each eye to select a different portion on each eye of the visual stimulus presented on the processing unit. This allows to vary the tests during the measurements. These can be red/green, yellow/blue or polarising filters to reproduce the 3-dimensional principle.

The periphery 18 of the opening 16 can be adapted to the patient, in particular to the contour of the face of the patient, the contour of the eyes of the patient and/or the presence of glasses on the face of the patient. This allows to avoid multiplying the size of the viewing cone, while still adapting to the patient. To do this, the cone comprises an adaptor that can be removably attached to the first spacer 221 (by clipping, for example). The adapter supports the periphery 18 and the opening 16 at the end 121 of the viewing cone. The adapter also has a standardised dimension, as do the spacers. The adaptor comprises one side attached to the spacer 221 and one side with a concave shape allowing to adapt to the morphology of the patient—child, adult, wide or thin face, presence or absence of glasses, etc. The advantage is that all you need is a series of adapters to fit any patient without having to multiply the number of spacers 221 formats—which reduces the overall dimension and the cost of the viewing cone.

The viewing cone 10 may further comprise a removable cover 28 adapted to obturate the opening 16, the cover 28 comprising a camera calibration pattern 30 on its side facing the interior of the chamber 12. This makes it quick and easy to calibrate the processing unit's camera. Such a calibration can be implemented by a practitioner in charge of measurements or by the patient himself. The calibration can be implemented quickly and easily for any distance between the periphery 18 of the opening 16 and the attachment interface 20 set to a fixed length. This also allows the camera to be calibrated independently of the patient. Usually, the camera calibration depends on the patient, who is asked to focus points on a screen and the position of the eyes is used as a reference for the calibration of the cameras. Sometimes, however, the patient is unable to perform this operation because they do not understand it or because their eyes are constantly moving. The pattern 30 on the cover 28 can be used to calibrate the camera in all these circumstances.

The fact that the distance between the periphery 18 of the opening 16 and the attachment interface 20 can be adjusted to a fixed length means that the calibration can be carried out in a standardised, predetermined environment with known dimensions. This environment is taken into account in the calibration and testing. The environment is standardised over time and even from one patient to another. In addition, the fact that adjustment to a fixed length is achieved by one or more spacers forming the wall and having a known and standardised dimension makes the calibration easier and more efficient.

The pattern 30, and possibly the cover 28, is also preferably curved. It can be curved towards the inside or outside of chamber 12, or in other words, concave or convex in relation to the chamber 12. The pattern 30 is not flat. This allows the camera to be calibrated with a three-dimensional pattern that has depth. This makes the calibration of the camera easier and more efficient. This is because, with a flat calibration pattern, it is not possible to solve the calibration equations; several images of the same plane must then be taken in different orientations to achieve this, which makes calibration more complex—or even renders it of poor quality or impossible if the calibration is carried out by the patient. The curved pattern 30 avoids such problems.

With the cover 28 obturating the opening 16, the pattern 30 is then positioned precisely on the cone 10 (directly on the cone) to ensure that it is positioned absolutely in relation to the position of the cameras. This provides an absolute reference for each of the points in the pattern 30. This gives the calibration algorithm an absolute position and orientation. For example, the pattern is precisely positioned on the cone using centring pins.

In addition, as the cover 28 obturates the opening 16, the pattern 30 is positioned in the same place as the eyes of the patient. This allows to immediately extract the distance between the two pupils (which can be useful, particularly in ophthalmology). This also allows the centre of the eyes to be extracted at any time. As the calibration is performed where the patient is looking at the processing unit, we obtain an estimate of the orientation of each eye with respect to a reference frame attached to the head.

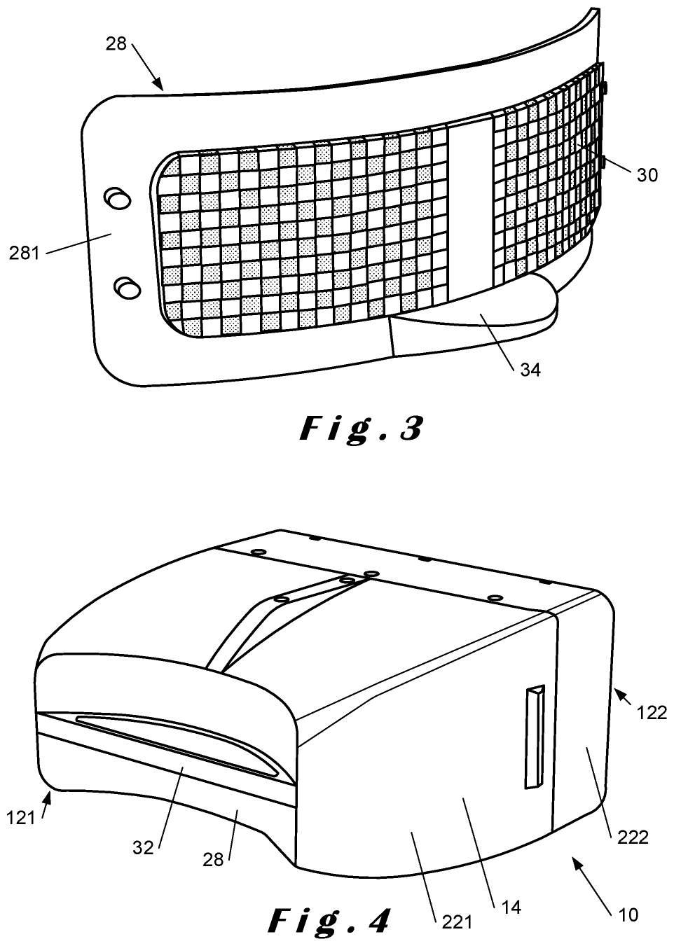

FIG. 3 shows the cover 28. The size of the cover 28 is such that the cover 28 obturates the opening 16 so as to completely darken the chamber 12. The shape of the cover 28 matches that of the periphery 18. The cover 28 may be flat (possibly with the pattern 30 curved (towards the inside or outside of the chamber 12, or in other words, concave or convex with respect to the chamber 12) on the side 281 of the cover) if the periphery 18 and the opening 16 are. The cover 28 is also preferably curved (towards the inside or the outside of the chamber 12, or in other words, concave or convex in relation to the chamber 12) to adapt to the concavity of the periphery 18, if necessary. The pattern 30 (and possibly the cover 28) is preferably concave, bulging towards the inside of the chamber 12. The pattern 30 is on the side 281 of the cover 28 intended to face the inside of the chamber 12. FIG. 4 shows the cover 28 in position on the viewing cone 10—the first spacer 221 in particular. The side 281 is turned towards the chamber 12 and the cover 28 is in place of the eyes of the patient; this allows a camera calibration without the viewing cone being in position on the patient. The pattern is, for example, a checkerboard, a black and white grid forming pixels corresponding to a known physical quantity. As the distance between the periphery 18 and the attachment interface 20 for attaching the processing unit is set to a fixed length, the distance between the eyes and the cameras is also known. This allows the camera to be calibrated in such a way that the gaze of the patient is correctly detected by the processing unit. The cover 28 may comprise a handle 32 on its side facing away from the chamber 12, such a handle makes it easier to position and remove the cover 28. In addition, the cover 28 can comprise a roughness 34 on the side facing the inside of the chamber 12. The asperity 34 allows to seal the cavity 19 in the periphery 18, adapting the viewing cone to the nose of the patient. The asperity 34 prevents the light from entering the chamber 12 during the camera calibration phase.

The viewing cone is a passive assembly. In other words, the structure of the viewing cone is able to operate without a power supply, independently of a power supply. The viewing cone, for example, is made of biocompatible, medical-grade plastic. The viewing cone 10 can be disinfected or sterilised between two patients. The viewing cone has no corners that are difficult or impossible to sterilise. The viewing cone is also hard-wearing and can be dropped from a height of 1 metre.

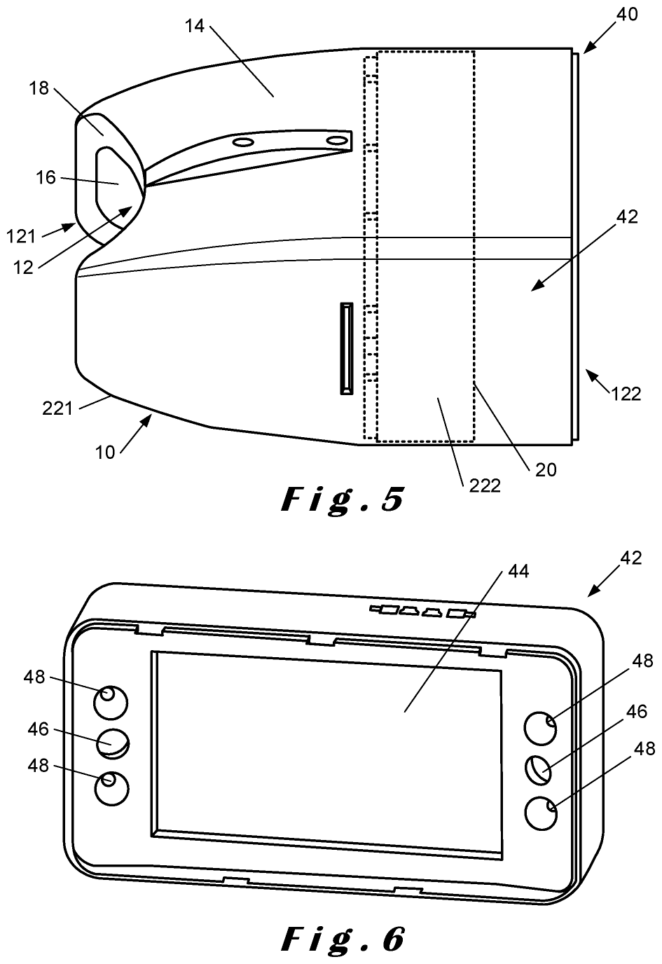

The invention also relates to a device 40 for detecting the movement of the eyes of a patient. According to an example shown in FIG. 5, the device 40 comprises the viewing cone 10 and a processing unit 42 attached to the attachment interface 20 of the vision cone. The processing unit 42 comprises cameras for tracking the movement of the eyes and a screen for displaying visual stimuli. The camera resolution is 640/480 pixels, for example. FIG. 6 shows an example of embodiment of the processing unit 42. The screen 44 occupies a large portion of a plane intended to be at the end 122 of the viewing cone. For example, a 15 cm diagonal screen allows tests to be carried out at +/−27° horizontal gaze variation and +/−16° vertical gaze variation. On either side of the screen 44, there are openings each designed to receive a camera 46 for detecting the movement of the eyes of the patient; the presence of two cameras means that one camera can be focused on each eye, thus increasing the spatial resolution. The cameras 46 are preferably at mid-height on either side of the screen 44 and therefore at eye level; this has the advantage of better detecting the movement of the eyes without re-processing the data to correct a possible angle with the plane containing the eyes. In addition, the processing unit 42 can receive emitters 48. These are preferably emitters in the infrared wavelength (e.g., 940 nm), not visible to the human eye, but allowing the eyes to be illuminated for the cameras 46 to detect the movement of the eyes. The processing unit 42 and therefore the device 40 also allow to make it easy to change the screen, the cameras and the emitters to increase the version.

According to FIG. 5, the distance between the periphery 18 of the opening 16 and the attachment interface 20 for attaching the processing unit 42 is adjustable to a fixed length by one or more spacers 221, 222, etc. forming the lateral wall 14. In this way, the distance between the face and the screen 44 of the processing unit 42 displaying the visual stimuli can be varied. By way of example, the viewing cone 10 comprises the first spacer 221 with the opening 16 at the first end 121 and comprises the second spacer 222 joined to the first spacer 221. The second end 122 with the attachment interface 20 is supported by the second spacer 222. The processing unit 42 is attached to the attachment interface 20. It is possible to have a single spacer 221—to which the processing unit 42 is attached—or two or more spacers, the spacers being assembled together. The processing unit 42 is attached to the attachment interface 20 supported by the last spacer.

The device 40 may be portable as described above with reference to the viewing cone 10 (by attaching the cone to the head of the patient) or may also comprise a support supporting the processing unit, the viewing cone being attached to the processing unit by the attachment interface 20. This allows to position the processing unit in an immobile manner. This also allows to lighten the device 40. The face of the patient then remains in the same position—which is also an advantage if the patient tends to move around too much. A strap described above allows to immobilise the face of the patient against the periphery 18. The support can be a stand that is placed on the floor or on a table; the support can also be an articulated arm, attached to a measuring bench for example.

The processing unit 42 can be connected to a computer of the practitioner to save the measurements during the series of measurements or even afterwards. Whether the device 40 is portable or supported by a support via the processing unit 42, the link to the computer can be made remotely or by a wired connection. The device 40 is therefore versatile.

The invention also relates to a method for detecting the movement of the eyes of a patient. The method comprises a step of providing the device 40 as previously described and a step of setting the distance between the periphery 18 of the opening 16 and the attachment interface 20 to a fixed length. This determines the depth of the chamber 12 in the direction of the gaze, between the face of the patient and the screen 44 of the processing unit 42. This makes it easy to carry out several series of measurements by varying the distance between the face and the screen 44.

In order to adjust the distance between the periphery 18 and the attachment interface 20—and therefore the distance between the face and the screen—the method may comprise a step in which one or more spacers 221, 222, etc. are used, interposed, added to form the lateral wall 14. The distance is easily adjustable and then remains fixed during the series of measurements, so that measurements can be taken in a standard environment.

Before starting a series of measurements, the method can also comprise a camera calibration step. During this step, the opening 16 is obturated by the removable cover 28 comprising the camera calibration pattern 30 on its side facing the inside of the chamber 12. This makes it easy for the practitioner or the patient to perform the calibration once the distance between the periphery 18 and the attachment interface 20 has been set to a fixed length. The environment defined by the viewing cone is standardised, predetermined and of known dimensions, making it easy to carry out the calibration and the testing. It is also quick and easy to recalibrate after changing the distance; several series of measurements can be taken without wasting time calibrating the camera. The calibration is easy, so the patient can carry out the tests at home without the presence of a practitioner.

The method can also comprise a step of stimulating the eyes by sending visual stimuli into the chamber 12 by the trough-orifices 24 in the lateral wall 14.

The method for detecting the movement of the eyes of a patient is therefore quick to set up, as it involves adjusting the distance between the eyes and the screen, calibrating the cameras, switching on the processing unit and eventually connecting to the computer of the practitioner.

The present invention has been described above in connection with specific embodiments, which are illustrative and should not be considered limiting. Generally speaking, it will be obvious to a person skilled in the art that the present invention is not limited to the examples illustrated and/or described above.

1. A viewing cone for detecting movement of eyes of a patient, the viewing cone comprising:
a chamber with a first end and a second end,
a lateral wall delimiting the chamber between the first end and the second end,
a periphery of an opening intended to be in contact with a forehead of the patient at the first end,
an attachment interface for attaching a processing unit at the second end,
a distance between the periphery of the opening and the attachment interface is adjustable to a fixed length by one or more spacers forming the lateral wall.

2. The viewing cone according to claim 1, comprising a single spacer supporting the periphery of the opening at the first end and the attachment interface for attaching the processing unit at the second end.

3. The viewing cone according to claim 1, comprising a first spacer supporting the periphery of the opening at the first end and one or more additional spacers joined together, the last spacer supporting the attachment interface for attaching the processing unit at the second end.

4. The viewing cone according to claim 1, wherein the spacers form between 5 and 15 cm of lateral wall.

5. The viewing cone according to claim 1, further comprising one or more through-orifices in the lateral wall, the through-orifices being adapted to send visual stimuli to different positions in the chamber.

6. The viewing cone according to claim 5, wherein the through-orifices are aligned between the first end and the second end.

7. The viewing cone according to claim 1, wherein the viewing cone is wearable on a face of the patient.

8. The viewing cone according to claim 1, comprising attachment members for attaching the cone to the head of the patient and/or handles.

9. The viewing cone according to claim 1, comprising a partition for a vision of each eye and/or a shutter for the vision of one of the eyes.

10. The viewing cone according to claim 9, wherein the shutter comprises a removable screen selectively obturating the vision of one eye or the other.

11. The viewing cone according to claim 1, comprising a filter in front of each eye to select a different portion on each eye of a visual stimulus presented by the processing unit.

12. The viewing cone according to claim 1, wherein the periphery of the opening is adaptable to a contour of a face of the patient and/or to the contour of the eyes and/or to a presence of glasses on the face of the patient.

13. The viewing cone according to claim 1, further comprising a removable cover adapted to obturate the opening, the removable cover comprising a camera calibration pattern on its side turned towards an inside of the chamber.

14. The viewing cone according to claim 13, wherein the camera calibration pattern is concave.

15. The viewing cone according to claim 1, wherein it is a passive assembly.

16. A device for detecting the movement of the eyes of a patient, comprising:
the viewing cone according to claim 1, and
a processing unit attached to the attachment interface of the viewing cone, the processing unit comprising cameras for tracking the movement of the eyes and a screen for displaying visual stimuli.

17. The device according to claim 16, wherein the distance between the periphery of the opening and the attachment interface for attaching the processing unit is adjustable to a fixed length by one or more spacers forming the lateral wall.

18. The device according to claim 16, wherein the viewing cone comprises a removable cover adapted to obturate the opening, the removable cover comprising a pattern for calibrating the cameras on its side turned towards an inside of the chamber.

19. The device according to claim 16, wherein the processing unit comprises infrared emitters illuminating the chamber.

20. The device according to claim 16, further comprising a support supporting the processing unit, the viewing cone being attached to the processing unit by the attachment interface.

21. A method for detecting the movement of eyes in a patient, comprising the steps of:
supplying the device according to claim 16, and
setting a distance between the periphery of the opening and the attachment interface (20) to a fixed length, one or more spacers are used to form the lateral wall.

22. The method according to claim 21, further comprising a step of calibrating the cameras during which the opening is obturated by a removable cover comprising a camera calibration pattern on its side turned towards an inside of the chamber.

23. The method of claim 22, wherein the camera calibration pattern is concave.

24. The method according to claim 21, further comprising a step of stimulating the eyes by sending visual stimuli into the chamber via through-orifices in the lateral wall.
